(19) United States
(12) Patent Application Publication
(10) Pub. No.: US 2004/0166169 A1
(43) Pub. Date: Aug. 26, 2004

(54) Porous extracellular matrix scaffold and method

(76) Inventors: Prasanna Malaviya, Fort Wayne, IN (US); Herbert Eugene Schwartz, Fort Wayne, IN (US); Pamela Lynn Plouhar, South Bend, IN (US); Mark Joseph Pelo, Macy, IN (US)

Correspondence Address: BARNES & THORNBURG, 11 SOUTH MERIDIAN, INDIANAPOLIS, IN, 46204

(21) Appl. No.: 10/483,930

(22) PCT Filed: Jul. 15, 2002
(86) PCT No.: PCT/US02/22393

(51) Int. Cl. A61K 35/37; A61K 35/38

(52) U.S. Cl. 424/551

(57) ABSTRACT

A method of making an implantable scaffold for repairing damaged or diseased tissue includes the step of suspending pieces of an extracellular matrix material in a liquid. The extracellular matrix material and the liquid are formed into a mass. The liquid is subsequently driven off so as to form interstices in the mass. Porous implantable scaffolds fabricated by such a method are also disclosed.

### CROSS REFERENCE TO RELATED APPLICATIONS

Cross reference is made to copending U.S. patent applications Ser. No. 10/195,794 entitled “Meniscus Regeneration Device and Method” (Attorney Docket No. 265280-71141, DEP-745); Ser. No. 10/195,719 entitled “Devices from Naturally Occurring Biologically Derived Materials” (Attorney Docket No. 265280-71142, DEP-748); Ser. No. 10/195,347 entitled “Cartilage Repair Apparatus and Method” (Attorney Docket No. 265280-71143, DEP-749); Ser. No. 10/195,344 entitled “Unitary Surgical Device and Method” (Attorney Docket No. DEP-750); Ser. No. 10/195,341 entitled “Hybrid Biologic/Synthetic Porous Extracellular Matrix Scaffolds” (Attorney Docket No. 265280-71144, DEP-751); Ser. No. 10/195,606 entitled “Cartilage Repair and Regeneration Device and Method” (Attorney Docket No. 265280-71145, DEP-752); Ser. No. 10/195,334 entitled “Cartilage Repair and Regeneration Scaffolds and Method” (Attorney Docket No. 265280-71180, DEP-763); and Ser. No. 10/195,633 entitled “Porous Delivery Scaffold and Method” (Attorney Docket No. 265280-71207, DEP-762), each of which is assigned to the same assignee as the present application, each of which is filed concurrently herewith, and each of which is hereby incorporated by reference. Cross reference is also made to U.S. patent application Ser. No. 10/172,347 entitled “Hybrid Biologic-Synthetic Bioabsorbable Scaffolds” which was filed on Jun. 14, 2002, which is assigned to the same assignee as the present application, and which is hereby incorporated by reference.

### FIELD OF THE DISCLOSURE

The present disclosure relates generally to an extracellular matrix scaffold, and more particularly to a porous extracellular matrix scaffold for repairing or regenerating body tissue and a method for making such a scaffold.

### BACKGROUND OF THE INVENTION

Naturally occurring extracellular matrices (ECMs) are used for tissue repair and regeneration. One such extracellular matrix is small intestine submucosa (SIS). SIS has been used to repair, support, and stabilize a wide variety of anatomical defects and traumatic injuries. Commercially available SIS material is derived from porcine small intestinal submucosa that remodels to the qualities of its host when implanted in human soft tissues. Further, it is taught that the SIS material provides a natural matrix with a three-dimensional microstructure and biochemical composition that facilitates host cell proliferation and supports tissue remodeling. Indeed, SIS has been shown to contain biological molecules, such as growth factors and glycosaminoglycans that aid in the repair of soft tissue of the human body. The SIS material currently being used in the orthopaedic field is provided in a dried and layered configuration in the form of a patch to repair or regenerate soft tissue such as tendons, ligaments and rotator cuffs.

While small intestine submucosa is readily available, other sources of ECM are known to be effective for tissue remodeling. These sources include, but are not limited to, stomach, bladder, alimentary, respiratory, or genital submucosa, or liver basement membrane. See, e.g., U.S. Pat. Nos. 6,379,710, 6,171,344; 6,099,567; and 5,554,389, each of which is hereby incorporated by reference.

Further, while SIS is most often porcine derived, it is known that various submucosa materials may also be derived from non-porcine sources, including bovine and ovine sources. Additionally, the ECM material may also include partial layers of laminar muscularis mucosa, muscularis mucosa, lamina propria, stratum compactum and/or other such tissue materials depending upon factors such as the source from which the ECM material was derived and the delamination procedure.

As used herein, it is within the definition of a naturally occurring extracellular matrix to clean, delaminate, and/or comminute the extracellular matrix, or to cross-link the collagen or other components within the extracellular matrix. It is also within the definition of naturally occurring extracellular matrix to fully or partially remove one or more components or subcomponents of the naturally occurring matrix. However, it is not within the definition of a naturally occurring extracellular matrix to separate and purify the natural components or subcomponents and reform a matrix material from purified natural components or subcomponents. Thus, while reference is made to SIS, it is understood that other naturally occurring extracellular matrices (e.g., stomach, bladder, alimentary, respiratory, and genital submucosa, and liver basement membrane), whatever the source (e.g., bovine, porcine, ovine) are within the scope of this disclosure. Thus, in this application, the terms “naturally occurring extracellular matrix” or “naturally occurring ECM” are intended to refer to extracellular matrix material that has been cleaned, processed, sterilized, and optionally crosslinked. The following U.S. patents, hereby incorporated by reference, disclose the use of ECMs for the regeneration and repair of various tissues: U.S. Pat. Nos. 6,379,710; 6,187,039; 6,176,880; 6,126,686; 6,099,567; 6,096,347; 5,997,575; 5,993,844; 5,968,096; 5,955,110; 5,922,028; 5,885,619; 5,788,625; 5,762,966; 5,755,791; 5,753,267; 5,733,337; 5,711,969; 5,645,860; 5,641,518; 5,554,389; 5,516,533; 5,460,962; 5,445,833; 5,372,821; 5,352,463; 5,281,422; and 5,275,826.

The manipulation of scaffold pore size, porosity, and interconnectivity is an important science contributing to the field of tissue engineering (Ma and Zhang, 2001, J Biomed Mater Res, 56(4):469-477; Ma and Choi, 2001 Tissue Eng, 7(1):23-33) because it is believed that the consideration of scaffold pore size and density/porosity influences the behavior of cells and the quality of tissue regenerated. In fact, several researchers have shown that different pore sizes influence the behavior of cells in porous three-dimensional matrices. For example, it has been demonstrated in the art that for adequate bone regeneration to occur scaffold pore size needs to be at least 100 microns (Klawitter et al., 1976, J Biomed Mater Res, 10(2):311-323). For pore sizes and interconnectivity less than that, poor quality bone is regenerated and if pore size is between 10-40 microns bone cells are able to form only soft fibro-vascular tissue (White and Shors, 1991, Dent Clin North Am, 30:49-67). The consensus of research for bone regeneration indicates that the requisite pore size for bone regeneration is 100-600 microns (Shors, 1999, Orthop Clin North Am, 30(4):599-613; Wang, 1990, Nippon Seikeigeka Gakki Zasshi, 64(9):847-859). It is generally known in the art that optimal bone regeneration occurs for pore sizes between 300-600 microns.

Similarly, for the regeneration of soft orthopaedic tissues, such as ligament, tendon, cartilage, and fibro-cartilage, scaffold pore size is believed to have a substantial effect. For example, basic research has shown that cartilage cells (chondrocytes) exhibit appropriate protein expression (type II collagen) in scaffolds with pore sizes of the order of 20 microns and tend to dedifferentiate to produce type I collagen in scaffolds with nominal porosity of about 80 microns (Nehrer et al., 1997, Biomaterials, 18(11):769-776). More recently, it has been shown that cells that form ligaments, tendons, and blood vessels (fibroblasts and endothelial cells) exhibit significantly different activity when cultured on scaffolds with differing pore sizes ranging from 5 to 90 microns (Salem et al., 2002, J Biomed Mater Res, 61(2):212-217).

### SUMMARY OF THE INVENTION

According to one illustrative embodiment, there is provided a method of making an implantable scaffold for repairing damaged or diseased tissue. The method includes the step of suspending, mixing, or otherwise placing pieces of a naturally occurring extracellular matrix material in a liquid. The naturally occurring extracellular matrix material and the liquid are formed into a mass. The liquid is subsequently driven off so as to form interstices in the mass. In one specific implementation of this exemplary embodiment, the liquid is driven off by freeze drying the naturally occurring extracellular matrix material and the liquid in which it is suspended. In such a manner, the liquid is sublimed thereby forming the interstices in the mass.

The material density and pore size of the scaffold may be varied by controlling the rate of freezing of the suspension. The amount of water into which the pieces of naturally occurring extracellular matrix material is suspended may also be varied to control the material density and pore size of the resultant scaffold.

In accordance with another exemplary embodiment, there is provided an implantable scaffold for repairing or regenerating tissue which is prepared by the process described above.

In another aspect, the present disclosure provides an implantable scaffold for repairing or regenerating body tissue. The scaffold comprises a porous body of naturally occurring extracellular matrix pieces that are interconnected to define an interior surface of the body. The interior surface has a three-dimensional topography of irregular shape.

In another aspect, the present disclosure provides an implantable device for repairing or regenerating body tissue. The device comprises a three-dimensional reticulated foam comprising a plurality of interconnected pores. The interconnected pores define three-dimensional interconnected passageways having irregular shapes. At least part of the reticulated foam comprises naturally occurring extracellular matrix.

In another aspect, the present disclosure provides a method of making an implantable device for repairing or regenerating body tissue. The method comprises the steps of providing a naturally occurring extracellular matrix material in a raw form, comminuting the raw naturally occurring extracellular matrix in the presence of a liquid to form a slurry of naturally occurring extracellular matrix, and lyophilizing the slurry of naturally occurring extracellular matrix to form a reticulated foam of naturally occurring extracellular matrix.

In another aspect, the present disclosure provides a method of making an implantable scaffold for repairing or regenerating body tissue. The method comprises the steps of providing a naturally occurring extracellular matrix material in a raw form, comminuting the raw naturally occurring extracellular matrix to form cohesive pieces of naturally occurring extracellular matrix, and lyophilizing the cohesive pieces of naturally occurring extracellular matrix to form a reticulated foam of naturally occurring extracellular matrix.

The implantable devices disclosed herein are three dimensional, porous scaffolds of ECMs like SIS. As such, it is evident that an implant based on the teachings of the present disclosure will have the dual advantage of having not only the appropriate biochemistry (collagens, growth factors, glycosaminoglycans, etc. naturally found in such ECMs) but also the appropriate physical microstructure to enable desired cellular activity upon implantation. These implantable devices are likely to find therapeutic use in the orthopaedic field, for devices used in the treatment of diseased or damaged fibro-cartilage such as the meniscus, diseased or damaged articular cartilage, and diseased or damaged bone.

The above and other features of the present disclosure will become apparent from the following description and the attached drawings.

### BRIEF DESCRIPTION OF THE DRAWINGS

The detailed description particularly refers to the accompanying figures in which:

FIG. 1 is an image from a scanning electron microscope which shows the surface of a porous reticulated SIS open cell foam scaffold having a relatively large pore size and a relatively low material density;

FIG. 2 is an image from a scanning electron microscope which shows the surface of a porous reticulated SIS open cell foam scaffold having a relatively moderate pore size and a relatively moderate material density;

FIG. 3 is an image from a scanning electron microscope which shows the surface of a porous reticulated SIS open cell foam scaffold having a relatively small pore size and a relatively high material density;

FIG. 4 is an image from a scanning electron microscope which shows a cross-section of a porous reticulated SIS open cell foam scaffold, with an example of a pore indicated by the arrow, the image being at a greater magnification than the images of FIGS. 1-3;

FIG. 5 is an image from a scanning electron microscope which shows a cross-section of a porous reticulated SIS open cell foam scaffold, with examples of pores indicated by the arrows, the image being at a greater magnification than the images of FIGS. 1-3;

FIG. 6 is an image from a scanning electron microscope which shows a cross-section of a porous reticulated SIS open cell foam scaffold, with examples of pores indicated by the arrows, the image being at a greater magnification than the images of FIGS. 1-3;

FIG. 7 is an image from a scanning electron microscope which shows a cross-section of a porous reticulated SIS open cell foam scaffold, the image being at a greater magnification than the images of FIGS. 1-3;

FIG. 8 is an image from a scanning electron microscope which shows a surface of a porous reticulated SIS open cell foam scaffold, the image being at a greater magnification than the images of FIGS. 1-3; and

FIGS. 9 and 10 are images from a scanning electron microscope which show a mass of cohesive SIS pieces.

### DETAILED DESCRIPTION OF THE DRAWINGS

The present disclosure relates to a porous scaffold for implanting into the body of a patient to repair or regenerate damaged or diseased tissue. The porous scaffold is constructed from a naturally occurring extracellular material. For example, the scaffold may be constructed from SIS. As will be discussed herein in greater detail, both the material density and the pore size of the porous scaffold may be varied to fit the needs of a given scaffold design.

Such porous scaffolds may be fabricated by suspending pieces of an extracellular matrix material in a liquid. As used herein, the term “piece” is intended to mean any fiber, strip, ribbon, sliver, filament, shred, bit, fragment, part, flake, slice, cut, chunk, or other portion of solid or solid-like material. Also, as used herein, the term “suspending” is intended to include any placement of a solid (e.g., pieces of ECM) in a liquid whether or not an actual suspension is created. As such, the term “suspending” is intended to include any mixing of a solid in a liquid or any other placement of a solid in a liquid. As a result, the term “suspension” is likewise not intended to be limited to suspensions, but rather is intended to mean any mass having a solid present in a liquid.

In any event, the suspension of the pieces of extracellular matrix material and the liquid forms a mass in the form of, for example, a “slurry”. The liquid may then be subsequently driven off of the mass so as to form interstices therein. The liquid may be driven off in a number of different manners. For example, as will herein be described in greater detail, the liquid may be driven off via sublimation in a freeze drying process. Alternatively, the liquid may also be driven off by subjecting the suspension to either an unheated vacuum process or a vacuum under a controlled heating process. The liquid may also be driven off from the suspension ultrasonically. Microwave energy, RF energy, UV energy, or any other type of energy (or combination thereof) may also be utilized to drive the liquid off of the suspension. Liquid may also be driven off of the suspension by forcing or drawing air through the suspension. The suspension may be centrifuged to drive off the liquid. Moreover, the liquid may include a water-soluble filler which is driven off, for example, by use of an alcohol. In short, the present disclosure contemplates the driving off of the liquid from the suspension by any liquid removal process.

As alluded to above, while any of the aforementioned processes for driving off the liquid from the suspension may be utilized, along with any other process known by one skilled in the art, the processes of the present disclosure will herein be exemplary described in regard to a lyophilization process (i.e., freeze drying). However, it should be understood that such a description is merely exemplary in nature and that any one or more of the aforedescribed processes for driving off the liquid from the suspension may be utilized to fit the needs of a given scaffold design or process design.

As alluded to above, one useful process for fabricating the porous scaffolds of the present disclosure is by lyophilization. In this case, pieces of an extracellular matrix material are suspended in a liquid. The suspension is then frozen and subsequently lyophilized. Freezing the suspension causes the liquid to be turned to ice crystals. These ice crystals are then sublimed under vacuum during the lyophilization process thereby leaving interstices in the material in the spaces previously occupied by the ice crystals. The material density and pore size of the resultant scaffold may be varied by controlling, amongst other things, the rate of freezing of the suspension and/or the amount of water in which the extracellular matrix material is suspended in at the on-set of the freezing process.

As a specific example of this process, fabrication of a porous SIS scaffold by lyophilization will be described in detail. However, it should be appreciated that although the example is herein described in regard to an SIS scaffold, fabrication of a scaffold constructed from other extracellular matrix materials may also be performed in a similar manner.

The first step in fabricating a porous scaffold with a desired pore size and density is the procurement of comminuted SIS. Illustratively, scissor-cut SIS runners (˜6″ long) are positioned in a 1700 series Comitrol™ machine, commercially available from Urschel Laboratories (Valparaiso, Ind.). The SIS material is processed in the presence of a liquid and thereafter collected in a receptacle at the output of the machine. The material is then processed through the machine a second time under similar conditions. In one exemplary process, a liquid (e.g., water) is introduced into the input of the machine contemporaneously with the SIS material. The resultant material is a “slurry” of SIS material (thin, long SIS fibers ˜200 microns thick×1-5 mm long) suspended in a substantially uniform manner in water. Although the suspension is herein described as being formed as a byproduct of the comminuting process, it should be appreciated that the pieces of SIS may be suspended in the liquid (i.e., water) in other manners known to those skilled in the art. Furthermore, while other methods are known for comminuting SIS, it is understood that for the purposes of the present disclosure, comminuted SIS comprises, ribbon-like or string-like fibers wherein at least some of the individual pieces of ECM and SIS material have lengths greater than their widths and thicknesses. Such fibers may be interlaced to provide a felt-like material, if desired.

Process parameters can be varied using the above-identified 1700 series Comitrol™ machine, including the choice of blade used, whether water is used, the amount of water used, the speed at which the blades turn, and the number of times the material is passed through the machine. As an example, cutting head 140084-10 and a Vericut, sealed impeller from Urschel Laboratories may be used, with a flow of water of about two (2) gallons per minute, with the blade running at a constant speed of about 9300 rpm. A first pass through the machine at these parameters will produce fibrous SIS material of varying sizes, and a second pass will produce SIS fibers of a more uniform size. By way of example, the comminuted material may be tested to determine if it has the consistency of that which is desired for use in regard to the illustrative embodiments described herein by the following process: the comminuted SIS suspension or slurry is centrifuged, excess water is poured off and the remaining slurry is poured into a dish. By hand, a small amount of the comminuted SIS material in the dish is pinched between the thumb and index finger and gently lifted from the dish. Illustratively, at least a small amount of additional SIS, beyond the portion pinched between the thumb and index finger, will lift along with the material that has been pinched (“pinch test”). This additional comminuted SIS material lifts with the material that is between the thumb and index finger because the individual pieces of comminuted SIS material are commingled or intertwined.

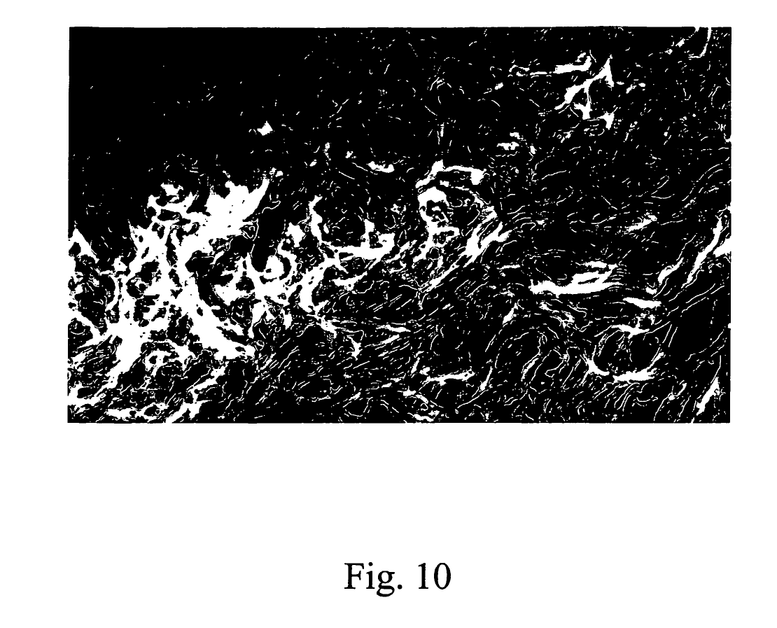

The terms “cohesive ECM”, “cohesive SIS”, “cohesive ECM pieces” and “cohesive SIS pieces” are used herein to respectively denote ECM or SIS material that has been comminuted or otherwise physically processed to produce ECM or SIS pieces that are capable of comingling or intertwining (in the wet or dry state) to form a mass of discrete pieces of ECM or SIS that remain massed together under some conditions (such as under gravity), regardless of the shape or shapes of the individual ECM or SIS pieces. One method of demonstrating that the ECM or SIS material comprises cohesive pieces is the “pinch test” described in the preceding paragraph. Examination of the final ECM or SIS product produced may also provide evidence that the base material comprised cohesive ECM or SIS pieces. Illustratively, the ECM or SIS pieces are sufficiently cohesive to each other (or to other pieces in the mix or slurry) that they remain unified throughout the process used to produce the foam structure. Examples of cohesive SIS pieces are shown in the scanning electron microscopic images of FIGS. 9 and 10.

Thereafter, the comminuted SIS suspension is frozen and lyophilized (i.e., freeze dried). In particular, the SIS suspension is frozen at a controlled rate of temperature drop to control the size of the formed ice crystals. Once frozen, and without allowing the material to thaw, the lyophilization process sublimes the ice crystals directly to a vapor under vacuum and low temperatures. This leaves voids or interstices in the spaces previously occupied by the ice crystals.

Any commercially available freezer for freezing the suspension to a desired temperature may be used. Likewise, any commercially available lyophilizer may be used for the lyophilization process. One exemplary machine for performing the lyophilization process is a Virtis Genesis™ Series lyophilizer which is commercially available from SP Industries, Inc. of Gardiner, N.Y.

The process parameters of the aforedescribed fabrication process may be varied to produce scaffolds of varying pore sizes and material densities. For example, the rate at which the suspension is frozen, the amount of water present in the suspension, or the compactness of the extracellular matrix material may be varied to produce scaffolds of varying pore sizes and material densities.

For instance, to produce scaffolds having a relatively large pore size and a relatively low material density, the extracellular matrix suspension may be frozen at a slow, controlled rate (e.g., −1° C./min or less) to a temperature of about −20° C., followed by lyophilization of the resultant mass. To produce scaffolds having a relatively small pore size and a relatively high material density, the extracellular matrix material may be tightly compacted by centrifuging the material to remove a portion of the liquid (e.g., water) in a substantially uniform manner prior to freezing. Thereafter, the resultant mass of extracellular matrix material is flash-frozen using liquid nitrogen followed by lyophilization of the mass. To produce scaffolds having a moderate pore size and a moderate material density, the extracellular matrix material is first tightly compacted by centrifuging the material to remove a portion of the liquid (e.g., water) in a substantially uniform manner prior to freezing. Thereafter, the resultant mass of extracellular matrix material is frozen at a relatively fast rate (e.g., >−1° C./min) to a temperature of about ˜80° C. followed by lyophilization of the mass.

### EXAMPLE 1

Example 1 demonstrates the fabrication of a porous SIS scaffold having a relatively large pore size and a relatively low material density. Such scaffolds are obtained by freezing a comminuted SIS suspension at a slow, controlled rate (−1° C./min or less) to −20° C., followed by lyophilization. The procedure is as follows. First, comminuted SIS is fabricated as described above. Specifically, scissor-cut SIS runners (˜6″ long) are placed in a suitable comminuting machine such as the Urschel Comitrol machine described above. The comminuted SIS is collected in a receptacle at the output of the machine. The collected material is then processed through the machine a second time, under the same conditions as before. The resultant mass is a “slurry” of SIS material (thin, long SIS fibers ˜200 microns thick×1-5 mm long) suspended relatively uniformly in water.

Next, a slow-freeze ethanol bath is prepared as follows. Pour enough ethanol to obtain about a 1 centimeter head in a flat-bottomed plastic container large enough to hold four 24-well culture plates. Place the container in a −20° C. freezer. The mass of each of four empty twenty-four well plates is then recorded. Under a sterile hood using sterile conditions, an approximately 3 ml aliquot of the comminuted SIS material is placed in each well of the tissue culture plates. The mass of each full plate of material is then recorded. The four culture plates are then placed into the ethanol freeze bath and allowed to freeze overnight.

The frozen plates are then removed from the ethanol bath and placed in a suitable lyophilization machine such as the Virtis Genesis Series lyophilizer described above. Without allowing the frozen SIS material to thaw, the process of lyophilization sublimes ice crystals directly to vapor under vacuum and low temperatures. This leaves voids or interstices in the spaces previously occupied by the ice crystals. In this case, the parameters used in the lyophilization process include a first period at a primary drying temperature of −13° C. for 8 hours, followed by a second period at a secondary drying temperature of 35° C. for 4 hours.

After the lyophilization cycle is complete, the plates are removed from the lyophilization machine and the mass of each plate is determined and recorded. The results from this process are summarized in the following table:

| Average Volume | Average Mass | Average Density |
|---|---|---|
| 1.249 ml | 0.007396 g | 0.006 (g/cc) |

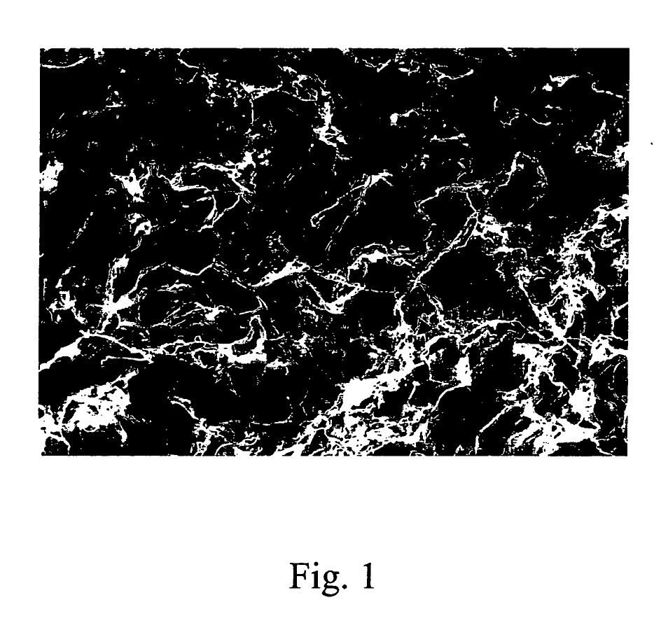

A scanning electron image of the surface of the samples was taken to visualize the relative pore sizes. These pore sizes are about 600 microns to about 700 microns. An image indicative of the samples prepared in accordance with Example 1 is shown in FIG. 1.

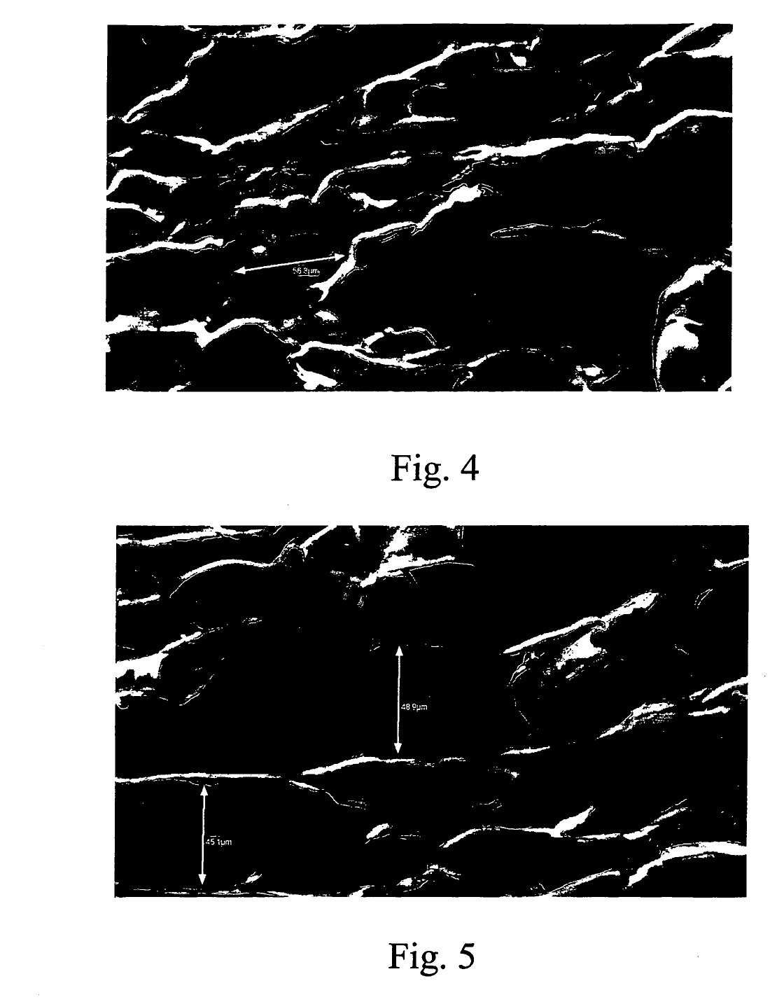

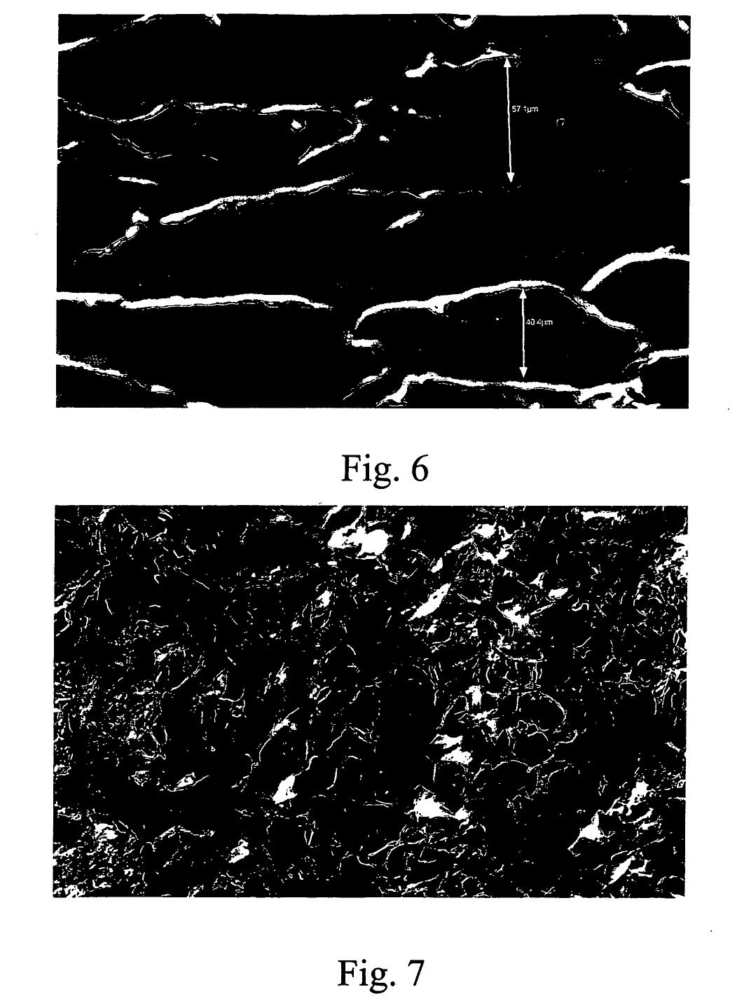

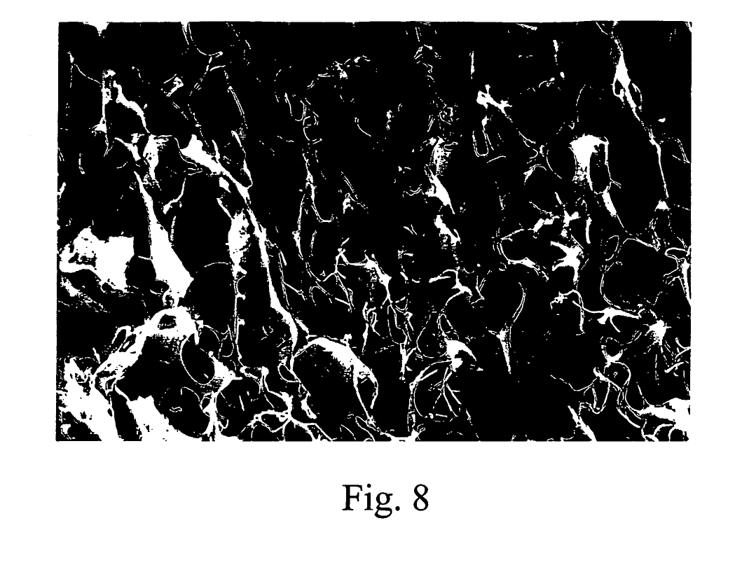

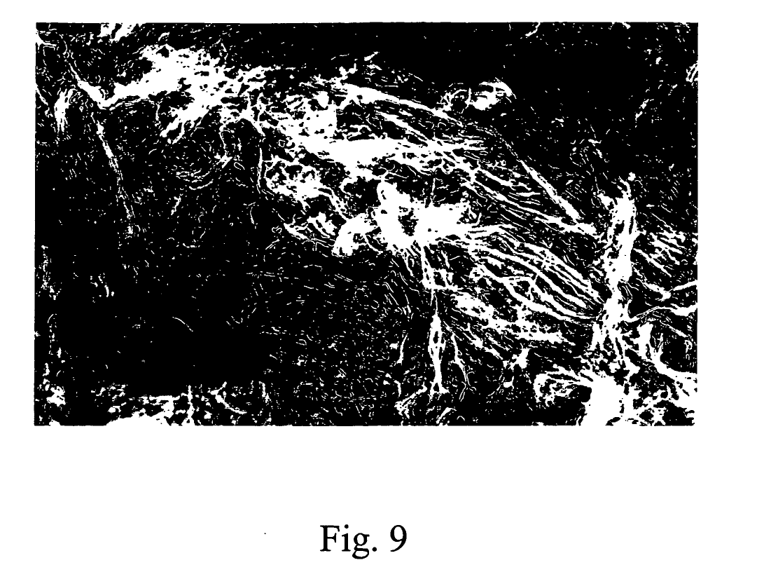

Pore sizes can be determined from scanning electron microscope images of the exterior surface of the foam, as in FIGS. 1-3 and 8, and of cross-sections of the foam, as in FIGS. 4-7. These images may be used in conjunction with standard commercially available image analysis software to determine the ranges of pore sizes. FIGS. 4-6 illustrate the results of using suitable commercially available software to measure or estimate the pore sizes in the foam. This technique was used to determine that the Example 1 foam had pores in the range of 600-700 microns. The sample may also include smaller pores.

### EXAMPLE 2

Example 2 demonstrates the fabrication of a porous SIS scaffold having a relatively moderate pore size and a relatively moderate material density. Such scaffolds are obtained by compacting the comminuted SIS material by centrifugation, freezing at a faster rate (relative to Example 1) and to a lower temperature (i.e., to −80° C.), followed by lyophilization of the resultant mass. The procedure is as follows. First, comminuted SIS is fabricated as described above in regard to Example 1. Specifically, scissor-cut SIS runners (˜6″ long) is comminuted by two passes through a suitable comminuting machine to produce a “slurry” of SIS material (thin, long SIS fibers ˜200 microns thick×1-5 mm long) suspended relatively uniformly in water.

Next, the mass of each of four empty twenty-four well plates is recorded. Under a sterile hood using sterile conditions, an approximately 3 ml aliquot of the comminuted SIS material is placed in each well of the tissue culture plates. The mass of each plate full of material is then recorded.

The plates are then balanced for centrifuging by use of the following technique. The two plates are placed on the balance, and RO water is added to the area in between the wells of the lighter plate until the two plates are balanced. The two plates are then placed across from one another in the centrifuge, and centrifuged at 3000 rpm for seven minutes. Once done, the plates are removed from the centrifuge, and the water is emptied therefrom. The mass of each of the plates is then recorded. The centrifuging and mass measurement process is then repeated for the remaining plates.

The plates are then placed in a −80° C. freezer until the specimen is fully frozen. Depending upon the bulk of the material, the time for full freezing can vary from about 1 to about 30 minutes, for example. The frozen plates are then removed from the freezer and placed in a suitable lyophilization machine and lyophilized under similar parameters to as described above in regard to Example 1 (i.e., for a first period at a primary drying temperature of −13° C. for 8 hours, followed by a second period at a secondary drying temperature of 35° C. for 4 hours).

After the lyophilization cycle is complete, the plates are removed from the lyophilization machine and the mass of each plate is determined and recorded. The results from this process are summarized in the following table:

| Average Volume | Average Mass | Average Density |
|---|---|---|
| 0.285 ml | 0.010104 g | 0.035 g/cc |

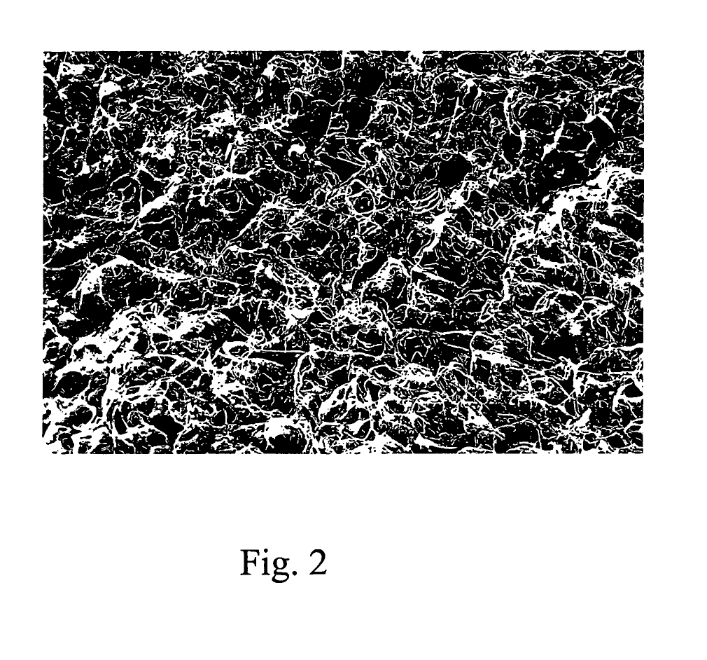

As with the samples of Example 1, a scanning electron image of the surface of each of the samples prepared in accordance with Example 2 was taken to visualize the relative pore sizes. An image indicative of the samples prepared in accordance with Example 2 is shown in FIG. 2. Using the technique described above for determining pore size, this sample was found to have pores in the range of about 100-150 microns.

### EXAMPLE 3

Example 3 demonstrates the fabrication of a porous SIS scaffold having a relatively small pore size and a relatively high material density. Such scaffolds are obtained by compacting the comminuted SIS material to an even higher density than in Example 2, flash-freezing the samples using liquid nitrogen, followed by lyophilization. The procedure is as follows. First, comminuted SIS is fabricated as described above in regard to Examples 1 and 2. Specifically, scissor-cut SIS runners (˜6″ long) is comminuted by two passes through a suitable comminuting machine to produce a “slurry” of SIS material (thin, long SIS fibers ˜200 microns thick×1-5 mm long) suspended relatively uniformly in water. Once done, the resultant mass is centrifuged under a dead-weight. The dead weights are prepared as follows. Forty-eight strips of Coban are cut into pieces that measure 50 mm in length and 5 mm in width (unstretched). Thereafter, the pieces are stretched and wrapped around the outer edges of a polyethylene disk measuring 1 cm in diameter and 2 mm in thickness. Each strip is trimmed, if need be, so that the Coban strips wrap around the disk two times.

Next, the mass of each of four empty twenty-four well plates is recorded. Under a sterile hood using sterile conditions, an approximately 3 ml aliquot of the comminuted SIS material is placed in each well of the tissue culture plates. The mass of each full plate of material is then recorded. The plates are then balanced and centrifuged as described above in regard to Example 2. Thereafter, the water is drained from the plates, and the mass of each of the centrifuged plates is recorded.

Once this is completed, the wells of the plates are combined at a ratio of 2:1 thereby reducing the number of plates from four to two. An attempt is made to combine low material wells with high material wells in order to have a somewhat consistent amount of SIS material in each well. The mass of each full plate is then recorded. The Coban-wrapped polyethylene disks are then placed into each well. The two plates are then balanced using the technique described above in regard to Example 2. The plates are then centrifuged at 3000 rpm for five minutes. Thereafter, the plates are removed from the centrifuge, the water is emptied therefrom, and the polyethylene disks are also removed. The mass of each plate is again recorded. The two plates are balanced once again (in a manner similar to as described above in regard to Example 2), and the plates are again centrifuged at 3000 rpm for seven minutes. Once done, the water is emptied again from each of the plates, and the mass of each plate is again recorded.

Contemporaneously with the centrifuging process, a liquid nitrogen bath is prepared by pouring liquid nitrogen into a wide-mouthed liquid nitrogen container. The plates are kept in the centrifuge until the bath is ready. Thereafter, each plate is dipped into the bath and held in the liquid for approximately 15 seconds. Upon removal from the nitrogen bath, the plates are immediately placed in a −80° C. freezer to prevent thawing. The frozen plates are then removed from the freezer and placed in a suitable lyophilization machine and lyophilized under similar parameters to as described above in regard to Examples 1 and 2 (i.e., for a first period at a primary drying temperature of −13° C. for 8 hours, followed by a second period at a secondary drying temperature of 35° C. for 4 hours).

After the lyophilization cycle is complete, the plates are removed from the lyophilization machine and the mass of each plate is determined and recorded. The results from this process are summarized in the following table:

| Average Volume | Average Mass | Average Density |
|---|---|---|
| 0.786 ml | 0.071563 g | 0.091 g/cc |

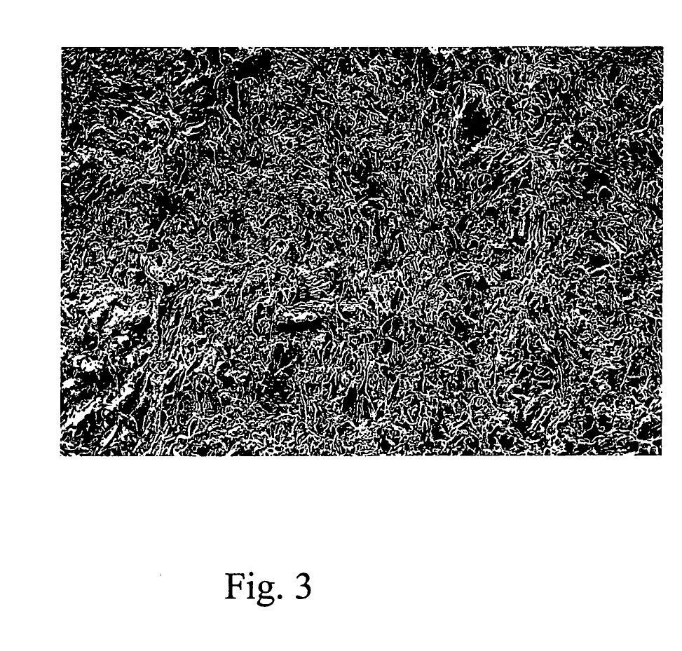

As with the samples of Examples 1 and 2, a scanning electron image of the surface of each of the samples prepared in accordance with Example 3 was taken to visualize the relative pore sizes. An image indicative of the samples prepared in accordance with Example 3 is shown in FIG. 3. Using the technique described above for determining pore size, this sample was found to have pores in the range of about 40-60 microns. Such pore sizes are illustrated in FIGS. 4-6, indicated by the arrows.

As shown in the scanning electron microscope images of FIGS. 1-8, each of the illustrated ECM foams comprises a three-dimensional network of reticulated naturally occurring ECM defining a plurality of interconnected pores. The foam has these pores throughout its height, width, and thickness. The pores are open and interconnected to define a plurality of irregularly shaped interconnected passageways leading from the exterior surface of the foam (see FIGS. 1-3 and 8) into the interior of the foam (see cross-sections FIGS. 4-7). These interconnected passageways are three-dimensional. As discussed above, the sizes of the pores, and therefore the maximum size for the interconnected passageways, can be controlled by controlling the manufacturing process as described above.

These interconnected passageways facilitate cell migration through the implant and enable efficient nutrient exchange in vivo. These interconnected passageways also provide a means of transmitting bioactive agents, biologically derived substances (e.g., stimulants), cells and/or biological lubricants, biocompatible inorganic materials, synthetic polymers and biopolymers (e.g., collagen) throughout the length, width and thickness of the ECM prior to implantation. The interconnected passageways defined by the pores also serve as passageways for materials used during the manufacturing process, such as compounds used for chemically cross-linking the foam. These interconnected passageways as well as the outer surfaces of the foam may also serve as sites on which the above materials are carried.

As shown in the above examples, the process parameters can be varied to produce an ECM foam that has the desired porosity for the particular application. For example, it may be desirable to produce a foam with lower density (and higher porosity) for applications involving osteocytes and to produce a foam with higher density (and lower porosity) for applications involving chondrocytes.

Moreover, the ECM foams described herein may be crosslinked. Specifically, the ECM foams described herein may be either chemically or physically crosslinked.

As can be seen in the scanning electron microscopic images of FIGS. 1-8, each of the illustrated ECM foams comprises interconnected pieces of naturally occurring extracellular matrix. As shown in the scanning electron microscope images of cross-sections of the ECM foams of FIGS. 4-7, these interconnected pieces of naturally occurring extracellular matrix provide the foam with an interior surface having an three-dimensional topography of irregular shape. As shown in the scanning electron microscope images of the surfaces of the ECM foam, these interconnected pieces of naturally occurring extracellular matrix provide the foam with exterior surfaces having three-dimensional topographies of irregular shapes. With these irregular three-dimensional topographies and the interconnected passageways, the ECM foams of the present disclosure provide a relatively large surface area of naturally occurring ECM. Such a large surface area of naturally occurring ECM can be advantageous in providing a relatively large surface area to which biological agents, biologically derived agents, cells, biocompatible polymers and biocompatible inorganic materials can be affixed pre-implantation. In addition, the illustrated ECM foams provide a relatively large surface of area of naturally occurring ECM to which cells may attach in vivo.

ECM foam products can be made with substantially lower densities than those of other ECM products. For comparison, the density of the commercially available RESTORE® product, an ECM laminate, is 0.466+/−0.074 g/cc. An ECM product consisting of comminuted and hardened SIS as described in U.S. patent application Ser. No. 10/195,719 entitled “Devices from Naturally Occurring Biologically Derived Materials” (Attorney Docket No. 265280-71142, DEP-748), has been made with a density of can be 0.747+/−0.059 g/cc. And, an ECM product consisting of toughened SIS laminate as described in U.S. patent application Ser. No. 10/195,794 entitled “Meniscus Regeneration Device and Method” (Attorney Docket No. 265280-7114, DEP-745) has been made with a density of 0.933+/−0.061 g/cc.

As discussed above, the ECM foams of the present disclosure may be combined with bioactive agents, biologically derived substances, cells and/or stimulants, biocompatible inorganic materials and/or biocompatible polymers (e.g., biocompatible synthetic polymers and biopolymers) and combinations of two or more of these materials at the time of manufacture. Illustratively, cells can be seeded throughout the three-dimensional volume of the ECM foam; the biological materials can be dried on the ECM foam at manufacture; the biological materials and the ECM foam can be co-lyophilized; and the biological materials can be covalently linked to the ECM foam. It is contemplated to bond, cross-link, or otherwise incorporate one or more of these materials to the raw ECM material prior to formation of the ECM foam. Alternatively, the materials could be bonded, cross-linked, or otherwise incorporated to the final ECM foam after lyophilization. Finally, combinations of the above methods may be used. For example, an implant of covalently linked ECM foam and biological lubricant can be implanted and additional intra-articular injections of the same or different biological lubricants can be made at surgery, post-operatively, or both at surgery and post-operatively.

“Bioactive agents” include one or more of the following: chemotactic agents; therapeutic agents (e.g., antibiotics, steroidal and non-steroidal analgesics and anti-inflammatories, anti-rejection agents such as immunosuppressants and anti-cancer drugs); various proteins (e.g., short chain peptides, bone morphogenic proteins, glycoprotein and lipoprotein); cell attachment mediators; biologically active ligands; integrin binding sequence; ligands; various growth and/or differentiation agents (e.g., epidermal growth factor, IGF-I, IGF-II, TGF-β I-III, growth and differentiation factors, vascular endothelial growth factors, fibroblast growth factors, platelet derived growth factors, insulin derived growth factor and transforming growth factors, parathyroid hormone, parathyroid hormone related peptide, bFGF; TGF_{β} superfamily factors; BMP-2; BMP-4; BMP-6; BMP-12; sonic hedgehog; GDF5; GDF6; GDF8; PDGF); small molecules that affect the upregulation of specific growth factors; tenascin-C; hyaluronic acid; chondroitin sulfate; fibronectin; decorin; thromboelastin; thrombin-derived peptides; heparin-binding domains; heparin; heparan sulfate; DNA fragments and DNA plasmids. If other such substances have therapeutic value in the orthopaedic field, it is anticipated that at least some of these substances will have use in concepts of the present disclosure, and such substances should be included in the meaning of “bioactive agent” and “bioactive agents” unless expressly limited otherwise.

“Biologically derived agents” include one or more of the following: bone (autograft, allograft, and xenograft) and derivates of bone; cartilage (autograft, allograft and xenograft), including, for example, meniscal tissue, and derivatives; ligament (autograft, allograft and xenograft) and derivatives; derivatives of intestinal tissue (autograft, allograft and xenograft), including for example submucosa; derivatives of stomach tissue (autograft, allograft and xenograft), including for example submucosa; derivatives of bladder tissue (autograft, allograft and xenograft), including for example submucosa; derivatives of alimentary tissue (autograft, allograft and xenograft), including for example submucosa; derivatives of respiratory tissue (autograft, allograft and xenograft), including for example submucosa; derivatives of genital tissue (autograft, allograft and xenograft), including for example submucosa; derivatives of liver tissue (autograft, allograft and xenograft), including for example liver basement membrane; derivatives of skin tissue; platelet rich plasma (PRP), platelet poor plasma, bone marrow aspirate, demineralized bone matrix, insulin derived growth factor, whole blood, fibrin and blood clot. Purified ECM and other collagen sources are also intended to be included within “biologically derived agents.” If other such substances have therapeutic value in the orthopaedic field, it is anticipated that at least some of these substances will have use in the concepts of the present disclosure, and such substances should be included in the meaning of “biologically derived agent” and “biologically derived agents” unless expressly limited otherwise.

“Biologically derived agents” also include bioremodelable collagenous tissue matrices. The expressions “bioremodelable collagenous tissue matrix” and “naturally occurring bioremodelable collagenous tissue matrix” include matrices derived from native tissue selected from the group consisting of skin, artery, vein, pericardium, heart valve, dura mater, ligament, bone, cartilage, bladder, liver, stomach, fascia and intestine, tendon, whatever the source. Although “naturally occurring bioremodelable collagenous tissue matrix” is intended to refer to matrix material that has been cleaned, processed, sterilized, and optionally crosslinked, it is not within the definition of a naturally occurring bioremodelable collagenous tissue matrix to purify the natural fibers and reform a matrix material from purified natural fibers. The term “bioremodelable collagenous tissue matrices” includes “extracellular matrices” within its definition.

“Cells” include one or more of the following: chondrocytes; fibrochondrocytes; osteocytes; osteoblasts; osteoclasts; synoviocytes; bone marrow cells; mesenchymal cells; stromal cells; stem cells; embryonic stem cells; precursor cells derived from adipose tissue; peripheral blood progenitor cells; stem cells isolated from adult tissue; genetically transformed cells; a combination of chondrocytes and other cells; a combination of osteocytes and other cells; a combination of synoviocytes and other cells; a combination of bone marrow cells and other cells; a combination of mesenchymal cells and other cells; a combination of stromal cells and other cells; a combination of stem cells and other cells; a combination of embryonic stem cells and other cells; a combination of precursor cells isolated from adult tissue and other cells; a combination of peripheral blood progenitor cells and other cells; a combination of stem cells isolated from adult tissue and other cells; and a combination of genetically transformed cells and other cells; If other cells are found to have therapeutic value in the orthopaedic field, it is anticipated that at least some of these cells will have use in the concepts of the present disclosure, and such cells should be included within the meaning of “cell” and “cells” unless expressly limited otherwise.

“Biological lubricants” include: hyaluronic acid and its salts, such as sodium hyaluronate; glycosaminoglycans such as dermatan sulfate, heparan sulfate, chondroiton sulfate and keratan sulfate; synovial fluid and components of synovial fluid, including as mucinous glycoproteins (e.g., lubricin), vitronectin, tribonectins, articular cartilage superficial zone proteins, surface-active phospholipids, lubricating glycoproteins I, II; and rooster comb hyaluronate. “Biological lubricant” is also intended to include commercial products such as ARTHREASE™ high molecular weight sodium hyaluronate, available in Europe from DePuy International, Ltd. of Leeds, England, and manufactured by Bio-Technology General (Israel) Ltd., of Rehovot, Israel; SYNVISC® Hylan G-F 20, manufactured by Biomatrix, Inc., of Ridgefield, N.J. and distributed by Wyeth-Ayerst Pharmaceuticals of Philadelphia, Pa.; HYLAGAN® sodium hyaluronate, available from Sanofi-Synthelabo, Inc., of New York, N.Y., manufactured by FIDIA S.p.A., of Padua, Italy; and HEALON® sodium hyaluronate, available from Pharmacia Corporation of Peapack, N.J. in concentrations of 1%, 1.4% and 2.3% (for opthalmologic uses). If other such substances have therapeutic value in the orthopaedic field, it is anticipated that at least some of these substances will have use in the concepts of the present disclosure, and such substances should be included in the meaning of “biological lubricant” and “biological lubricants” unless expressly limited otherwise.

“Biocompatible polymers” is intended to include both synthetic polymers and biopolymers (e.g., collagen). Examples of biocompatible polymers include: polyesters of [alpha]-hydroxycarboxylic acids, such as poly(L-lactide) (PLLA) and polyglycolide (PGA); poly-p-dioxanone (PDO); polycaprolactone (PCL); polyvinyl alcohol (PVA); polyethylene oxide (PEO); polymers disclosed in U.S. Pat. Nos. 6,333,029 and 6,355,699; and any other bioresorbable and biocompatible polymer, co-polymer or mixture of polymers or co-polymers that are utilized in the construction of prosthetic implants. In addition, as new biocompatible, bioresorbable materials are developed, it is expected that at least some of them will be useful materials from which orthopaedic devices may be made. It should be understood that the above materials are identified by way of example only, and the present invention is not limited to any particular material unless expressly called for in the claims.

“Biocompatible inorganic materials” include materials such as hydroxyapatite, all calcium phosphates, alpha-tricalcium phosphate, beta-tricalcium phosphate, calcium carbonate, barium carbonate, calcium sulfate, barium sulfate, polymorphs of calcium phosphate, ceramic particles, and combinations of such materials. If other such substances have therapeutic value in the orthopaedic field, it is anticipated that at least some of these substances will have use in the concepts of the present disclosure, and such substances should be included in the meaning of “biocompatible inorganic material” and “biocompatible inorganic materials” unless expressly limited otherwise.

It is expected that various combinations of bioactive agents, biologically derived agents, cells, biological lubricants, biocompatible inorganic materials, biocompatible polymers can be used with the scaffolds of the present disclosure.

It is expected that standard sterilization techniques may be used with the products of the present disclosure.

Illustratively, in one example of embodiments that are to be seeded with living cells such as chondrocytes, a sterilized implant may be subsequently seeded with living cells and packaged in an appropriate medium for the cell type used. For example, a cell culture medium comprising Dulbecco's Modified Eagles Medium (DMEM) can be used with standard additives such as non-essential aminoacids, glucose, ascorbic acid, sodium pyrovate, fungicides, antibiotics, etc., in concentrations deemed appropriate for cell type, shipping conditions, etc.

It is anticipated that the ECM foams of the present disclosure may be combined with the concepts disclosed in the following applications for U.S. patent, filed concurrently herewith, which are incorporated by reference herein in their entireties: Ser. No. 10/195,794 entitled “Meniscus Regeneration Device and Method” (Attorney Docket No. 265280-71141, DEP-745); Ser. No. 10/195,719 entitled “Devices from Naturally Occurring Biologically Derived Materials” (Attorney Docket No. 265280-71142, DEP-748); Ser. No. 10/195,347 entitled “Cartilage Repair Apparatus and Method” (Attorney Docket No. 265280-71143, DEP-749); Ser. No. 10/195,344 entitled “Unitary Surgical Device and Method” (Attorney Docket No. DEP-750); Ser. No. 10/195,341 entitled “Hybrid Biologic/Synthetic Porous Extracellular Matrix Scaffolds” (Attorney Docket No. 265280-71144, DEP-751); Ser. No. 10/195,606 entitled “Cartilage Repair and Regeneration Device and Method” (Attorney Docket No. 265280-71145, DEP-752); Ser. No. 10/195,334 entitled “Cartilage Repair and Regeneration Scaffolds and Method” (Attorney Docket No. 265280-71180, DEP-763); and Ser. No. 10/195,633 entitled “Porous Delivery Scaffold and Method” (Attorney Docket No. 265280-71207, DEP-762), along with U.S. patent application Ser. No. 10/172,347 entitled “Hybrid Biologic-Synthetic Bioabsorbable Scaffolds” which was filed on Jun. 14, 2002. For example, for orthopaedic uses, it may be desirable to accompany or follow implantation with a treatment regime involving administering hyaluronic acid to the implantation site.

As can be seen from the forgoing description, the concepts of the present disclosure provide numerous advantages. For example, the concepts of the present disclosure provide for the fabrication of a porous implantable scaffold which may have varying mechanical properties to fit the needs of a given scaffold design. For instance, the pore size and the material density may be varied to produce a scaffold having a desired mechanical configuration. In particular, such variation of the pore size and the material density of the scaffold is particularly useful when designing a scaffold which provides for a desired amount of cellular migration therethrough, while also providing a desired amount of structural rigidity. In addition, according to the concepts of the present disclosure, implantable devices can be produced that not only have the appropriate physical microstructure to enable desired cellular activity upon implantation, but also has the biochemistry (collagens, growth factors, glycosaminoglycans, etc.) naturally found in such ECMs.

Although it is believed that naturally occurring extracellular matrix provides advantages over purified extracellular matrix, it is contemplated that the teachings of the present disclosure can be applied to purified extracellular matrix as well. Thus, it is expected that the naturally occurring extracellular matrix could be purified prior to physically comminuting the extracellular matrix. This purification could comprise treating the naturally occurring extracellular matrix to remove substantially all materials other than collagen prior to physically comminuting the extracellular matrix. The purification could be carried out to substantially remove glycoproteins, glycosaminoglycans, proteoglycans, lipids, non-collagenous proteins and nucleic acids (DNA and RNA).

While the disclosure is susceptible to various modifications and alternative forms, specific exemplary embodiments thereof have been shown by way of example in the drawings and has herein be described in detail. It should be understood, however, that there is no intent to limit the disclosure to the particular forms disclosed, but on the contrary, the intention is to cover all modifications, equivalents, and alternatives falling within the spirit and scope of the disclosure.

There are a plurality of advantages of the present disclosure arising from the various features of the apparatus and methods described herein. It will be noted that alternative embodiments of the apparatus and methods of the present disclosure may not include all of the features described yet still benefit from at least some of the advantages of such features. Those of ordinary skill in the art may readily devise their own implementations of an apparatus and method that incorporate one or more of the features of the present disclosure and fall within the spirit and scope of the present disclosure.

1. A method of making an implantable scaffold for repairing or regenerating body tissue, the method comprising the steps of:
suspending pieces of a naturally occurring extracellular matrix material in a liquid; and
freeze drying the pieces of naturally occurring extracellular matrix material and the liquid.

2. The method of claim 1, further comprising the step of freezing the extracellular matrix material and the liquid to form ice crystals from the liquid, the freezing step being performed prior to the freeze drying step.

3. The method of claim 2, wherein the freeze drying step further comprises subliming the ice crystals directly to vapor in the presence of a vacuum.

4. The method of claim 1, wherein the freeze drying step comprises subliming the liquid so as to form a porous body.

5. The method of claim 1, further comprising the step of comminuting the extracellular matrix material into the pieces.

6. The method of claim 1, wherein the extracellular matrix material comprises material selected from the group consisting of: small intestine submucosa, bladder submucosa, stomach submucosa, alimentary submucosa, respiratory submucosa, genital submucosa, and liver basement membrane

7. The method of claim 1, further comprising the step of flash-freezing the extracellular matrix material and the liquid prior to the freeze drying step.

8. The method of claim 1, further comprising the step of compacting the pieces of naturally occurring extracellular matrix material prior to the freeze drying step.

9. The method of claim 1, further comprising the step of centrifuging the pieces of naturally occurring extracellular matrix material prior to the freeze drying step.

10. The method of claim 1, further comprising the step of freezing the naturally occurring extracellular matrix material and the liquid at a controlled rate of temperature drop.

11. The method of claim 10, wherein the freezing step comprises varying the rate of temperature drop so as to vary the pore size of the scaffold.

12. The method of claim 1, further comprising the step of adding at least one of the following: a bioactive agent; a biologically derived agent; cells; a biological lubricant; a biocompatible inorganic material; and a biocompatible polymer.

13. A method of making an implantable scaffold for repairing or regenerating body tissue, the method comprising the steps of:
suspending pieces of a naturally occurring extracellular matrix material in a liquid;
forming the pieces of the naturally occurring extracellular matrix and the liquid into a mass; and
driving off the liquid so as to form interstices in the mass.

14. The method of claim 13, wherein the driving off step comprises subliming the liquid.

15. The method of claim 13, wherein the driving off step comprises vaporizing the liquid.

16. The method of claim 13, further comprising the steps of:
providing a naturally occurring extracellular matrix in a raw form, and
comminuting the naturally occurring extracellular matrix material to form the pieces of naturally occurring extracellular matrix, the pieces of naturally occurring extracellular matrix being smaller than the raw form of the naturally occurring extracellular matrix.

17. The method of claim 13, wherein the naturally occurring extracellular matrix material comprises material selected from the group consisting of: small intestine submucosa, stomach submucosa, respiratory submucosa, alimentary submucosa, genital submucosa, bladder submucosa, and liver basement membrane.

18. The method of claim 13, wherein the forming step comprises compacting the pieces of naturally occurring extracellular matrix material.

19. The method of claim 13, further comprising the step of adding at least one of the following: a bioactive agent; a biologically derived agent; cells; a biological lubricant; a biocompatible inorganic material; and a biocompatible polymer.

20. An implantable scaffold for repairing or regenerating body tissue, comprising a porous body which is prepared by a process comprising the steps of:
suspending pieces of a naturally occurring extracellular matrix material in a liquid, and
freeze drying the naturally occurring extracellular matrix material and the liquid.

21. The implantable scaffold of claim 20, wherein the process for preparing the porous body further comprises the step of freezing the naturally occurring extracellular matrix material and the liquid to form ice crystals from the liquid, the freezing step being performed prior to the freeze drying step.

22. The implantable scaffold of claim 21, wherein the freeze drying step further comprises subliming the ice crystals directly to vapor in the presence of a vacuum.

23. The implantable scaffold of claim 20, wherein the freeze drying step comprises subliming the liquid.

24. The implantable scaffold of claim 20, wherein the process for preparing the porous body further comprises the step of comminuting the naturally occurring extracellular matrix material into the pieces.

25. The implantable scaffold of claim 20, wherein the naturally occurring extracellular matrix material comprises material selected from the group consisting of: small intestine submucosa, stomach submucosa, bladder submucosa, alimentary submucosa, respiratory submucosa, genital submucosa, and liver basement membrane.

26. The implantable scaffold of claim 20, wherein the process for preparing the porous body further comprises the step of flash-freezing the naturally occurring extracellular matrix material and the liquid prior to the freeze drying step.

27. The implantable scaffold of claim 20, wherein the process for preparing the porous body further comprises the step of compacting the pieces of naturally occurring extracellular matrix material prior to the freeze drying step.

28. The implantable scaffold of claim 20, wherein the process for preparing the porous body further comprises the step of centrifuging the pieces of naturally occurring extracellular matrix material prior to the freeze drying step.

29. The implantable scaffold of claim 20, further comprising at least one of the following: a bioactive agent; a biologically derived agent; cells; a biological lubricant; a biocompatible inorganic material; and a biocompatible polymer.

30. An implantable scaffold for repairing or regenerating body tissue, comprising: porous body which is prepared by a process comprising the steps of:
suspending pieces of a naturally occurring extracellular matrix material in a liquid,
forming the pieces of the extracellular matrix and the liquid into a mass, and
driving off the liquid so as to form interstices in the mass.

31. The implantable scaffold of claim 30, wherein the driving off step comprises subliming the liquid.

32. The implantable scaffold of claim 30, wherein the driving off step comprises vaporizing the liquid.

33. The implantable scaffold of claim 30, wherein the process for preparing the porous scaffold further comprises the step of comminuting the naturally occurring extracellular matrix material into the pieces.

34. The implantable scaffold of claim 30, wherein the naturally occurring extracellular matrix material comprises material selected from the group consisting of: small intestine submucosa, stomach submucosa, bladder submucosa, alimentary submucosa, respiratory submucosa, genital submucosa, and liver basement membrane.

35. The implantable scaffold of claim 30, wherein the forming step comprises compacting the pieces of naturally occurring extracellular matrix material.

36. The implantable scaffold of claim 30, further comprising at least one of the following: a bioactive agent; a biologically derived agent; cells; a biological lubricant; a biocompatible inorganic material; and a biocompatible polymer.

37. An implantable scaffold for repairing or regenerating body tissue, the scaffold comprising:
a porous body of naturally occurring extracellular matrix pieces interconnected to define an interior surface having a three-dimensional topography of irregular shape.

38. The implantable scaffold of claim 37, wherein:
the interconnected naturally occurring extracellular matrix pieces define interstices in the body, and
the interstices are sized by mixing the matrix pieces with water to form a moistened mass and freezing the water in a controlled manner to control the size of the frozen water crystals, thereby controlling the size of the interstices.

39. The implantable scaffold of claim 37, wherein at least part of the interconnected naturally occurring extracellular matrix pieces defines pores having a nominal pore size of 100-700 microns.

40. The implantable scaffold of claim 39, wherein at least part of the interconnected naturally occurring extracellular matrix pieces defines pores having a nominal pore size of 300-700 microns.

41. The implantable scaffold of claim 37, wherein at least part of the interconnected naturally occurring extracellular matrix pieces defines pores having a nominal pore size of less than 100 microns.

42. The implantable scaffold of claim 37, further comprising at least one of the following: a bioactive agent; a biologically derived agent; cells; a biological lubricant; a biocompatible inorganic material; and a biocompatible polymer.

43. An implantable device for repairing or regenerating body tissue, the device comprising a three-dimensional-open cell foam comprising a plurality of interconnected pores defining three-dimensional interconnected passageways having irregular shapes, at least part of the foam comprising naturally occurring bioremodelable collagenous tissue matrix.

44. The implantable device of claim 43, wherein the foam comprises interconnected pieces of naturally occurring bioremodelable collagenous tissue matrix.

45. The implantable device of claim 43, wherein the foam has an interior surface, said interior surface comprising naturally occurring bioremodelable collagenous tissue matrix having an irregularly shaped three-dimensional topography.

46. The implantable device of claim 43, further comprising at least one of the following: a bioactive agent; a biologically derived agent; cells; a biological lubricant; a biocompatible inorganic material; and a biocompatible polymer.

47. The implantable device of claim 43, wherein the naturally occurring bioremodelable collagenous tissue matrix comprises at least a portion of at least one of the following: small intestine submucosa, stomach submucosa, bladder submucosa, alimentary submucosa, respiratory submucosa, genital submucosa and liver basement membrane.

48. The implantable device of claim 43, wherein the nominal pore size of at least part of the device is between 100-700 microns.

49. The implantable device of claim 43, wherein the nominal pore size of at least part of the device is between 300-700 microns.

50. The implantable device of claim 43, wherein the nominal pore size of at least part of the device is less than 100 microns.

51. The implantable device of claim 43, wherein the foam has a density of about 0.005-0.5 g/cc.

52. An implantable device for repairing or regenerating body tissue, the device comprising a porous reticulated body of naturally occurring bioremodelable collagenous tissue matrix.

53. The implantable device of claim 52, wherein the naturally occurring bioremodelable collagenous tissue matrix comprises at least a portion of at least one of the following: small intestine submucosa, stomach submucosa, bladder submucosa, alimentary submucosa, respiratory submucosa, genital submucosa and liver basement membrane.

54. The implantable device of claim 52, further comprising at least one of the following: a bioactive agent; a biologically derived agent; cells; a biological lubricant; a biocompatible inorganic material; and a biocompatible polymer.

55. The implantable device of claim 52, wherein at least part of the reticulated body defines pores having a nominal pore size of 100-700 microns.

56. The implantable device of claim 52, wherein at least part of the reticulated body defines pores having a nominal pore size of 300-700 microns.

57. The implantable device of claim 52, wherein at least part of the reticulated body defines pores having a nominal pore size of less than 100 microns.

58. The implantable device of claim 52, wherein at least part of the reticulated body defines a plurality of interconnected pores defining three-dimensional interconnected passageways having irregular shapes.

59. A method of making an implantable device for repairing or regenerating body tissue, the method comprising the steps of:
providing a naturally occurring extracellular matrix material in a raw form;
comminuting the raw naturally occurring extracellular matrix in the presence of a liquid to form a slurry of naturally occurring extracellular matrix; and
lyophilizing the slurry of naturally occurring extracellular matrix to form an open cell foam of naturally occurring extracellular matrix.

60. The method of claim 59 wherein the open cell foam of naturally occurring extracellular matrix includes molecules other than collagen, said molecules other than collagen being present in the raw form of the naturally occurring extracellular matrix.

61. The method of claim 59, further comprising adding at least one of the following: a bioactive agent; a biologically derived agent; cells; a biological lubricant; a biocompatible inorganic material; and a biocompatible polymer.

62. A method of making an implantable scaffold for repairing or regenerating body tissue, the method comprising the steps of:
providing a naturally occurring bioremodelable collagenous tissue matrix in a raw form;
comminuting the raw naturally occurring bioremodelable collagenous tissue matrix to form cohesive pieces of naturally occurring bioremodelable collagenous tissue matrix; and
lyophilizing the cohesive pieces of naturally occurring bioremodelable collagenous tissue matrix to form a reticulated foam of naturally occurring bioremodelable collagenous tissue matrix.

63. The method of claim 62, wherein the reticulated foam of naturally occurring bioremodelable collagenous tissue matrix includes molecules other than collagen, said molecules other than collagen being present in the raw form of the naturally occurring extracellular matrix.

64. The method of claim 62, further comprising adding at least one of the following: a bioactive agent; a biologically derived agent; cells; a biological lubricant; a biocompatible inorganic material; and a biocompatible polymer.

65. A method of making an implantable scaffold for repairing or regenerating body tissue, the method comprising the steps of:
providing a extracellular matrix material derived from tissue selected from the group consisting of: small intestine submucosa, stomach submucosa, bladder submucosa, respiratory submucosa, alimentary submucosa, genital submucosa, and liver basement membrane;
physically comminuting the extracellular matrix to form cohesive pieces of extracellular matrix; and
lyophilizing the cohesive pieces of extracellular matrix to form an open cell foam of extracellular matrix.

66. The method of claim 65, wherein the extracellular matrix comprises naturally occurring extracellular matrix, and the step of physically comminuting the extracellular matrix comprises physically comminuting the naturally occurring extracellular matrix.

67. The method of claim 65, further comprising purifying the extracellular matrix prior to physically comminuting the extracellular matrix.

68. The method of claim 65, further comprising treating the extracellular matrix to substantially remove glycoproteins, glycosaminoglycans, proteoglycans, lipids, non-collagenous proteins and nucleic acids.

69. The method of claim 65, further comprising treating the extracellular matrix to remove substantially all materials other than collagen prior to physically comminuting the extracellular matrix.

70. The method of claim 65, wherein the step of physically comminuting the extracellular matrix comprises physically comminuting the extracellular matrix in the presence of a liquid.

71. A product made by the process of claim 65.

72. The product of claim 71 further comprising at least one of the following: a bioactive agent; a biologically derived agent; cells; a biological lubricant; a biocompatible inorganic material; and a biocompatible polymer.

73. A method of making an implantable device for repairing or regenerating body tissue, the method comprising the steps of:
providing a extracellular matrix material selected from the group consisting of: small intestine submucosa, stomach submucosa, bladder submucosa, respiratory submucosa, alimentary submucosa, genital submucosa, and liver basement membrane; and
physically comminuting the extracellular matrix in the presence of a liquid to form a slurry of extracellular matrix.

74. The method of claim 73, further comprising lyophilizing the slurry of extracellular matrix to form a reticulated foam of extracellular matrix.

75. The method of claim 73, wherein the extracellular matrix material comprises naturally occurring extracellular matrix material and wherein the step of physically comminuting the extracellular matrix in the presence of a liquid comprises physically comminuting naturally occurring extracellular matrix in the presence of a liquid.

76. The method of claim 73, further comprising purifying the extracellular matrix prior to physically comminuting the extracellular matrix.

77. The method of claim 73, further comprising treating the extracellular matrix to substantially remove glycoproteins, glycosaminoglycans, proteoglycans, lipids, non-collagenous proteins and nucleic acids such as DNA and RNA.

78. A product made according to the method of claim 66.

79. The product of claim 78 further comprising at least one of the following: a bioactive agent; a biologically derived agent; cells; a biological lubricant; a biocompatible inorganic material; and a biocompatible polymer.

80. A method of making an implantable scaffold for repairing or regenerating body tissue, the method comprising the steps of:
providing an extracellular matrix material selected from the group consisting of: small intestine submucosa, stomach submucosa, bladder submucosa, respiratory submucosa, alimentary submucosa, genital submucosa, and liver basement membrane;
suspending pieces of the extracellular matrix material in a liquid; and
freeze drying the pieces of extracellular matrix material and the liquid.

81. The method of claim 80, further comprising adding at least one of the following: a bioactive agent; a biologically derived agent; cells; a biological lubricant; a biocompatible inorganic material; and a biocompatible polymer.

82. A method of making an implantable scaffold for repairing or regenerating body tissue, the method comprising the steps of:
providing an extracellular matrix material selected from the group consisting of: small intestine submucosa, stomach submucosa, bladder submucosa, respiratory submucosa, alimentary submucosa, genital submucosa and liver basement membrane;
suspending pieces of the extracellular matrix material in a liquid;
forming the pieces of the naturally occurring extracellular matrix and the liquid into a mass; and
driving off the liquid so as to form interstices in the mass.

83. The method of claim 82, further comprising adding at least one of the following: a bioactive agent; a biologically derived agent; cells; a biological lubricant; a biocompatible inorganic material; and a biocompatible polymer.

84. A slurry comprising cohesive pieces of naturally occurring extracellular matrix in a liquid.

85. A slurry of extracellular matrix material comprising cohesive pieces of extracellular matrix in a liquid, the extracellular matrix material being selected from the group consisting of: small intestine submucosa, stomach submucosa, bladder submucosa, respiratory submucosa, alimentary submucosa, genital submucosa, and liver basement membrane.

86. A method of treating defective cartilage in a joint of a patient comprising:
implanting a biocompatible device in the patient at the joint;
the device comprising a foam comprising naturally occurring extracellular matrix having a plurality of interconnected pores, at least part of the extracellular matrix having interconnected pores having a nominal pore size of between about 30 and 100 microns.

87. A method of treating defective cartilage in a joint of a patient comprising:
providing an extracellular matrix material selected from the group consisting of: small intestine submucosa, stomach submucosa, bladder submucosa, respiratory submucosa, alimentary submucosa, genital submucosa, and liver basement membrane;
forming the extracellular matrix material into a foam having a plurality of interconnected pores, at least part of the extracellular matrix having interconnected pores having a nominal pore size of between 30 and 100 microns; and
implanting the foam at the joint of the patient.

88. A method of treating diseased or damaged bone comprising:
implanting a device in the diseased or damaged bone;
the device comprising a foam comprising naturally occurring extracellular matrix having a plurality of interconnected pores, at least part of the extracellular matrix having interconnected pores having a nominal pore size greater than about 200 microns.

89. A method of treating diseased or damaged bone comprising:
providing an extracellular matrix material selected from the group consisting of: small intestine submucosa, stomach submucosa, bladder submucosa, respiratory submucosa, alimentary submucosa, genital submucosa, and liver basement membrane;
forming the extracellular matrix material into a foam having a plurality of interconnected pores, the interconnected pores having an irregular shape and extending in three dimensions, at least part of the extracellular matrix having interconnected pores having a nominal pore size greater than 200 microns; and
implanting the foam in the bone having the disease or damage.

90. An implantable device for repairing or regenerating body tissue, the device comprising a porous naturally occurring extracellular matrix, the porous matrix having a density of less than 0.1 g/cc.

91. The implantable device of claim 90 wherein the porous matrix has a density of less than 0.04 g/cc.

92. The implantable device of claim 91 wherein the porous matrix has a density of less than 0.01 g/cc.

93. The implantable device of claim 90 wherein the porous naturally occurring extracellular matrix comprises SIS.

94. The implantable device of claim 90 wherein the porous matrix comprises a reticulated foam.

95. The implantable device of claim 90 wherein the porous matrix comprises an open cell foam.

96. The implantable device of claim 90 wherein the porous matrix comprises a plurality of pores defining three-dimensional interconnected passageways having irregular shapes.

97. A method of processing an extracellular matrix material comprising the step of comminuting the extracellular matrix material in the presence of a liquid.

98. The method of claim 97, wherein:
the extracellular matrix material comprises small intestine submucosa, and
the comminuting step comprises comminuting the small intestine submucosa in the presence of the liquid.

99. The method of claim 97, wherein:
the liquid comprises water, and
the comminuting step comprises comminuting the extracellular matrix material in the presence of water.
